(19) United States
(12) Patent Application Publication
(10) Pub. No.: US 2011/0264012 A1
(43) Pub. Date: Oct. 27, 2011

(54) COMPLIANT COUPLANT WITH LIQUID RESERVOIR FOR TRANSDUCER

(76) Inventors: Frans Lautzenhiser, Indianapolis, IN (US); Fred Grossman, Indianapolis, IN (US); Glen Boehm, Winchester, IN (US)

(21) Appl. No.: 12/909,250

(22) Filed: Oct. 21, 2010

Related U.S. Application Data
(60) Provisional application No. 61/254,483, filed on Oct. 23, 2009.
(60) Provisional application No. 61/314,340, filed on Mar. 16, 2010.

(51) Int. Cl. A61N 7/00

(52) U.S. Cl. 601/2

(57) ABSTRACT

An elastomeric pad structure coupled to an ultrasonic transducer provides excellent coupling and transmission for extradermal applications without requiring the glycerin gel couplant typically used for such applications. The material and shape of the face of the pad structure traps a small amount of liquid between the pad and the skin, providing an effective couplant not requiring further containment, rewetting, or clean-up. The elastomeric pad be releasably coupled to the ultrasonic transducer, and the various illustrative embodiments for coupling include the use of clips, adhesives, an adapter ring having a lip or grooves, a ring having pins for engaging holes in the elastomeric pad and the transducer, and a sleeve which employs pressure or friction to retain the elastomeric pad to the transducer. Uses for the system and method include extradermal applications such as ultrasonic bone growth stimulation through a window cut in a bone fracture cast.

### CROSS-REFERENCE TO RELATED APPLICATIONS

This application claims the benefit of U.S. Provisional Patent Application Nos. 61/254,483 and 61/314,340, filed Oct. 23, 2009, and Mar. 16, 2010, respectively, which are incorporated herein by reference.

### BACKGROUND

The present invention relates to a gel-free system for ultrasonic transducers, and more particularly, to a couplant face for ultrasonic transducers used for extradermal applications.

Ultrasonic transducers used for extradermal applications are well known and usually have either a flat or a smoothly-curved face. In contrast, even the smoothest skin is covered with small features such as pores or hairs which prevent uniform contact with the face of the transducer. As a result, an area of air exists between the face of the transducer and the patch of skin where the two are not in direct contact. Because of the large acoustic impedance mismatch between air and the face of a transducer, there is essentially no ultrasonic energy transmitted to the subdermal target except when the transducer face is in direct, complete contact with the skin.

To avoid the problem of incomplete transfer, generally a “couplant” is used to fill the volume between the skin and the ultrasonic transducer face and improve the transfer of the acoustic wave from the transducer to the target. The acoustic impedance of the couplant is similar to that of flesh so that the ultrasonic energy will readily transmit through the interface between couplant and the skin. Commonly used couplants include water, mineral oil, and glycerin gel. While water or mineral oil are safe and have good acoustic properties for the application, they have the disadvantage that they will readily flow away from the interface and so must be submerged or replenished to ensure consistent contact. To avoid this problem, glycerin gel is most often used because it is hypoallergenic and because its viscoelastic properties prevent the gel from simply flowing away during use.

Despite the effectiveness of glycerin gel, there are drawbacks to this couplant. The glycerin gel does not evaporate away after use, but dries into a crusty mass. This is an issue for applications where cleaning away the glycerin gel after use is problematic, e.g., when the ultrasonic transducer is placed through an opening or window in a cast, as is often the case for ultrasonic bone growth stimulation. Additionally, for ultrasonic imaging, the use of a transducer and optional standoff with gel provide a more slippery connection with the skin than is desired, especially for biopsy or other procedures.

### SUMMARY

The present invention comprise one or more of the features recited in the attached claims and/or one or more of the following features and combinations thereof.

A face pad structure for coupling with the face of an ultrasonic transducer provides compliance with the skin and allows a small amount of water to work as an effective couplant. One illustrative face pad structure according to the present disclosure includes an elastomeric, or compliant, material. The face pad include a raised perimeter lip which is formed from a soft elastomeric material. The face pad structure include a back face having a convex surface. The elastomeric material preferably has a low attenuation to minimize signal loss in the pad and acoustic impedance similar to that of water and skin to avoid excessive reflection at the interface. The durometer (mechanical stiffness) of the elastomeric material preferably is low so that the perimeter lip and interior face of the pad can readily conform to the surface of the skin without the need to apply excessive pressure.

By itself, the elastomeric pad is effectively a dry couplant structure. However, even very soft pads will not completely fill pores and surround hairs as would be needed for complete coupling and thus transmission of the acoustic wave generated by the ultrasonic transducer. The use of water or another impedance-matched liquid with a dry couplant pad improves the coupling initially, but such liquids are susceptible to draining away during the contact period, leading to reduced transmission of acoustic energy to the target. The use of structural features, for example the raised perimeter lip, to trap the water between the skin and the pad face allows improved and sustained performance of the coupling.

An illustrative ultrasonic transducer system includes a transducer body and an elastomeric pad. The transducer body includes a piezoelectric element and a final matching layer defining a transducer face. The elastomeric pad includes a back face coupled with the transducer face and a front face contoured to retain a liquid such as water between the front face and a target surface, for example, skin. The front face include a rim protruding axially adjacent the perimeter of the front face. The front face define a concave surface, a stepped depression, or an angled recess. The front face have a diameter about equal to the diameter of the transducer face. The front face or another portion of the elastomeric pad include a porous material or reservoir for retaining a liquid. The elastomeric pad be formed from the elastomeric copolymer styrene-ethylene/butylene-styrene (SEBS), silicone, for example silicone rubber, or other elastomeric materials that are hypoallergenic, have an acoustic impedance closely matching that of skin, high compliance (low durometer), and low acoustic dissipation.

A thin member of SEBS can be torn and also is difficult to adhere to other materials, thus, in one illustrative ultrasonic transducer system, the elastomeric pad is cast onto a matrix which provides structural integrity for the pad and/or structural coupling to the ultrasonic transducer. The matrix include a screen, for example, formed from woven cloth, woven or molded polyester, or other material providing a support structure with minimal acoustic dissipation.

In another illustrative ultrasonic transducer system, the elastomeric pad includes one or more flanges axially protruding from the perimeter of the back face, the one or more flanges being anchored to the transducer body. The system include one or more retaining members adapted to anchor the flanges to the transducer body.

In another illustrative ultrasonic transducer system, the system includes a structure containing a rewetting solution. The structure include a cup and a rewetting pad positioned inside the cup, the cup and rewetting pad arranged so that the transducer be positioned with the front face of the elastomeric pad in contact with the rewetting pad. Absorption of the rewetting solution into the elastomeric pad extends the useful life of the elastomeric pad. The structure also include a filter positioned between the elastomeric pad and the rewetting pad, for example, a filter that provides unidirectional flow of solution from the rewetting pad to the elastomeric pad. The structure also include one or more retaining members for retaining the transducer, including the elastomeric pad, against the rewetting pad. The structure also include a seal for preventing the solution from evaporating or otherwise escaping the cup. The solution include mineral oil.

In yet another illustrative ultrasonic transducer system, the elastomeric pad is releasably coupled to the ultrasonic transducer. One illustrative embodiment involves an adapter ring having multiple pins for engaging corresponding holes in an elastomeric pad and holes in an ultrasonic transducer while other embodiments using an adapter include, but are not limited to, a sleeve which employs pressure or friction to couple an elastomeric pad to an ultrasonic transducer, an elastomeric pad which is fitted to an ultrasonic transducer by one or more clips, and an elastomeric pad which is adhesively bonded to a transducer body, with or without the use of an adapter ring.

In another illustrative ultrasonic transducer system, the elastomeric pad includes a pharmaceutical to be delivered by contact with skin and the stimulation of transfer by ultrasound.

Additional features of the disclosure will become apparent to those skilled in the art upon consideration of the following detailed description of the illustrative embodiment.

### BRIEF DESCRIPTION OF THE DRAWINGS

The detailed description particularly refers to the accompanying figures in which:

FIGS. 1A and 1B illustrates a cross-sectional view of prior art ultrasonic transducers in a typical extradermal application;

FIG. 2A illustrates a cross-sectional view of an illustrative ultrasonic transducer system according to the present invention in an extradermal application;

FIG. 2B illustrates a perspective view of the illustrative ultrasonic transducer system of FIG. 2B

FIG. 3A illustrates a cross-sectional view of an another illustrative ultrasonic transducer system according to the present invention;

FIG. 3B illustrates a perspective view of the illustrative ultrasound transducer system of FIG. 3A;

FIGS. 4A-4B illustrate disassembled components of the illustrative ultrasonic transducer system of FIG. 3A;

FIG. 5A illustrates the elastomeric pad and adapter ring of the illustrative ultrasonic transducer system of FIG. 3A;

FIG. 5B illustrates cross-sectional view of a illustrative fixture for molding the elastomeric pad and adapter ring of FIG. 5A;

FIGS. 6A-6C illustrate acoustic wave reflection test results for the illustrative ultrasonic transducer system of FIG. 3A coupled to air, water, and skin, respectively;

FIGS. 7A-7B illustrate test results for transmitted energy versus time for the illustrative ultrasonic transducer system of FIG. 3A coupled to skin without water and with water, respectively;

FIGS. 8A-8B respectively illustrate an exploded and an assembled cross-sectional view of another illustrative ultrasonic transducer system according to the present invention;

FIG. 9 illustrates a cross-sectional view of the illustrative alternative sonic transducer system of FIGS. 8A-8B contained within a rewetting cup according to the present invention;

FIG. 10A illustrates a perspective view of a ring-and-pin device for connecting an elastomeric pad to an ultrasonic transducer;

FIG. 10B illustrates a cross-sectional view of the ring-and-pin device of FIG. 10A;

FIG. 11 illustrates a cross-sectional view of the ring-and-pin device of FIG. 10A coupled with an elastomeric pad and ultrasonic transducer;

FIG. 12 illustrates a cross-sectional view of a friction sleeve for retaining an elastomeric pad and ultrasonic transducer;

FIG. 13 illustrates a cross-sectional view of an ultrasonic transducer coupled to an elastomeric pad having a concave recess;

FIG. 14 illustrates a cross-sectional view of an ultrasonic transducer coupled to an elastomeric pad having a stepped recess;

FIG. 15 illustrates a cross-sectional view of a an elastomeric pad which is clipped onto an ultrasonic transducer;

FIG. 16 illustrates a cross-sectional view of an elastomeric pad which is coupled to an ultrasonic transducer through the use of an adapter ring; and

FIG. 17 illustrates an exploded perspective view of the adapter ring and the housing of the ultrasonic transducer of FIG. 16.

### DESCRIPTION OF THE ILLUSTRATIVE EMBODIMENTS

For the purposes of promoting and understanding the principals of the invention, reference will now be made to one or more illustrative embodiments shown in the drawings and specific language will be used to describe the same.

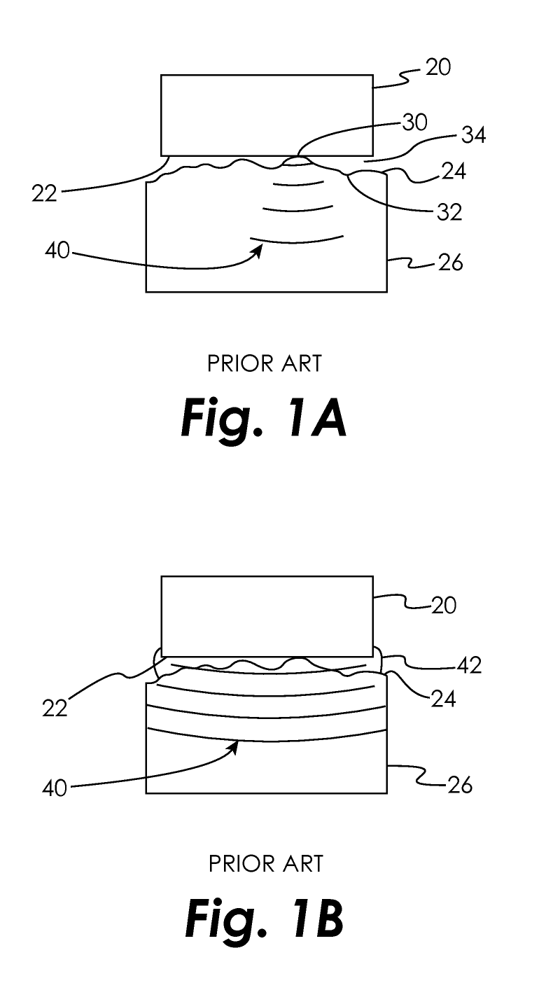

Referring to FIGS. 1A and 1B, a cross-sectional view of a prior art ultrasonic transducer 20 positioned relative to a subdermal target 26 in an extradermal application is illustrated. In FIG. 1A, the ultrasonic transducer 20 has a rigid front face 22, generally comprising an impedance matching layer. The outer skin 24 overlying a subdermal target region 26 provides a non-uniform contact area with the transducer face 22, thus providing areas of direct contact 30, and areas of no contact 32 where the skin 24 and transducer face 22 are separated by air 34, hair, or debris (not shown). Because the impedance matching layer of the front face 22 is matched for skin 24, rather than air 34, the transmitted acoustic wave 40 is highly attenuated and is much less than is desired.

To overcome the poor transmission, as shown in FIG. 1B, generally a layer of couplant 42 is positioned between the transducer face 22 and the skin 24 above the subdermal target 26 and the couplant 42. Typical couplants 42 include water, mineral oil, and glycerin gel. Because the couplant 42 has a substantially improved impedance match with the skin 24 and matching layer of the transducer face 22 than does air 34, the amplitude of the transmitted acoustic wave 40 is greatly improved over that when no couplant 42 is used, as in FIG. 1A.

The disadvantage to using typical couplants 42 is that liquids and gels either readily flow or are squeezed away from the interface between the ultrasonic transducer 20 and the skin 24 over the subdermal target 26. Although glycerin gel has viscoelastic properties that prevent the gel from simply flowing away as water or mineral oil does, such gel still provide undesirable drawbacks including the required cleanup after it dries into a crusty mass. In the case of ultrasonic bone growth stimulation applications, in which a ultrasonic transducer is usually positioned against the skin through a window cut in a cast, the gel escapes from under the transducer face 22 and is pushed in between the cast and skin in the areas surrounding the window cut in the cast, making cleanup difficult, if not impossible, without removing the cast.

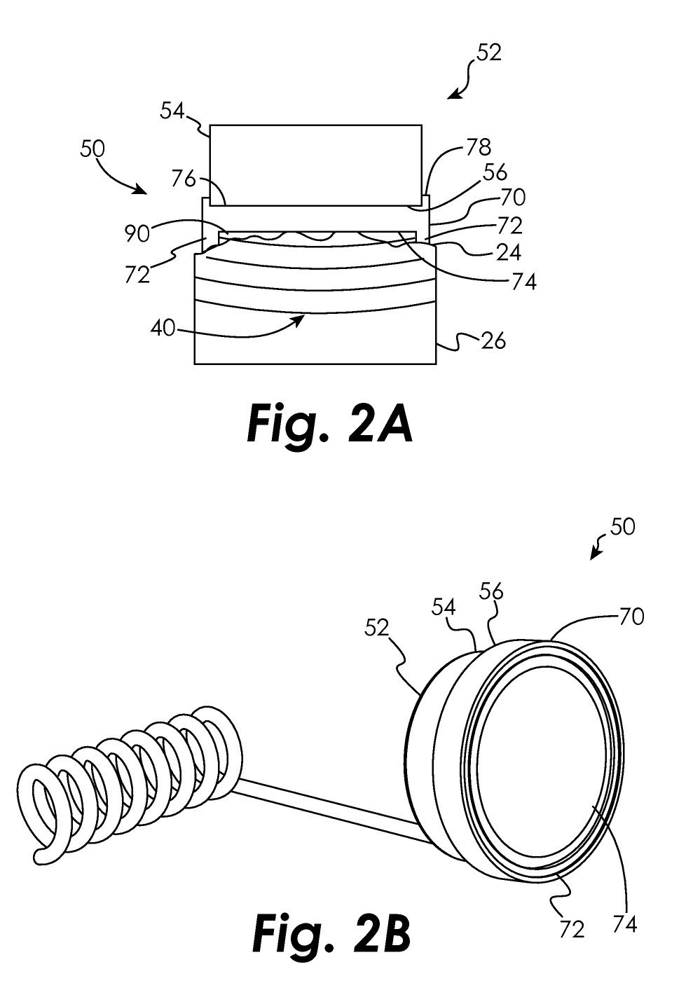

Referring to FIG. 2A, an illustrative ultrasonic transducer system 50 according to the present disclosure is being used in an extradermal application, for example, bone growth stimulation. The ultrasonic transducer system 50 (also shown in FIG. 2B) includes an ultrasonic transducer 52 and an elastomeric pad 70. Compliant elastomeric pad 70 provides an acoustic impedance closely matching that of the skin 24 above the subdermal target 26 and further includes a structural feature to trap a liquid 90, such as water, between the pad 70 and skin 24. The structural feature used in ultrasonic transducer system 50 includes a lip 72 that axially protrudes adjacent the perimeter of the front face 74 of the elastomeric pad 70. The structural feature also or alternatively include the front face 74 having a recess which is concave, stepped, or angled in contour.

The elastomeric pad 70 includes a back face 76 in direct contact with the front face 56 of the ultrasonic transducer 52. The elastomeric pad 70 also include one or more flanges 78 or other features that provide mechanical coupling or other releasable or permanent bonding with the transducer body 54. In the embodiment of releasable coupling of the elastomeric pad 70 to the transducer 52, the transducer 52 be reusable and the elastomeric pad 70 be designed for a single, disposable use, for example, with less durable structural integrity. Exemplary designs could omit a screen or other matrix to provide structural support and coupling to the transducer 52.

The lack of intervening air and acoustic impedance matching of the ultrasonic transducer 52, pad 70, water 90, and skin 24 provides excellent transmission of acoustic wave 40 from the ultrasonic transducer 52 into the skin 24 and toward the subdermal target 26.

The elastomeric pad 70 be formed from a styrene-based elastomeric material, such as the elastomeric copolymer styrene-ethylene/butylene-styrene (SEBS) or other elastomeric materials, in particular silicones such as silicone rubber. The elastomeric material preferably is hypoallergenic, has an acoustic impedance (preferably at least 1.0 acoustic ohms, more preferably at least about 1.15) reasonably matching that of skin (about 1.5 acoustic ohms), has high compliance (low durometer, for example a 15 to 25 Shore 00), and a low acoustic dissipation (preferably less than 10 decibels per centimeter). In one illustrative ultrasonic transducer system 50, the elastomeric pad 70 is cast from three parts of SEBS DS-302, available from California Medical Innovations, of Pomona, Calif., and one part mineral oil USP, which is widely available.

Other suitable materials for forming the elastomeric pad 70 include soft elastomers such as rubbers with a low glass transition temperature. The rubbers contain oil to reduce the glass transition temperature, but preferably do not contain fillers or voids. An illustrative example of this material comprises: high cis-butadiene rubber, such as that available from National Petrochemical Co., Iran, or EniChem S.P.A.; a crosslinker such as Dicumyl Peroxide; and a low viscosity naphthenic oil, such as Strukthene 410 available from Safic Alcan. In one example, the composition is 100 pph high cis-butadiene rubber, 1 pph Dicumyl Peroxide, and 900 pph Strukthene 410, while in another illustrative example, only 400 pph Strukthene 410 is used. More specifically, this material be prepared, without any heat being required, by the following steps: cutting the rubber into small pieces, adding the oil, stirring with a z-blade mixer until the mixture is homogeneous, adding the crosslinker, and casting and covering the mixture while it cures.

Alternatively, the elastomeric pad 70 material be composed of 1 part Kraton G1650/1652 (copolymer based on styrene/ethylene/butadiene) with 9 parts light paraffin oil (C8-C10 alkanes).

In another illustrative composition, the material needed for the elastomeric pad 70 is prepared by the following steps: an oil loaded polyurethane elastomer is prepared by placing Desmodur N3200 (23.4 g) in a reaction vessel and a polyethylene glycol-polypropylene glycol monobutyl ether random copolymer (176.6 g, MW 3900) is added together with bismuth tris neodecanoate/decanoic acid catalyst (0.04 g, Coscat 83). The mixture is heated at 70° C. for 6 hours to form a pre-polymer. A portion of this pre-polymer (55.0 g) is placed in a reaction vessel and a polyethylene glycol-polypropylene glycol-polyethylene glycol block copolymer (20.0 g) is added together with Coscat 83 (0.004 g), and the mixture is stirred at room temperature for 30 seconds. Diethylene glycol dibutyl ether (300 g) is added, and the mixture is stirred for 10 seconds and poured into a glass dish. The resulting reaction mixture is cured at 70° C. for 12 hours.

In another illustrative composition, the material needed for the elastomeric pad 70 is prepared by the following steps: an oil loaded polyurethane elastomer is prepared by the following method: Desmodur E305 (16.6 g) is placed in a reaction vessel and Levagel VPKA 8732 (83.09 g) is added together with Coscat 83 catalyst. The reaction mixture is stirred for 1 minute. Diethylene glycol dibutyl ether (300 g) is added, and the mixture is stirred for 10 seconds and poured into a glass dish. The resulting reaction mixture is cured at 70° C. for 12 hours.

Dipropylene glycol dimethyl ether can also be used in place of diethylene glycol dibutyl ether. Desmodur 3200 is an isocyanate terminated polyether pre-polymer on a base of hexamethylene diisocyanate (2 functional). Levagel VPKA 8732 is a polyether polyhydric alcohol (4 functional). The elastomeric pad material also be prepared by swelling the oil into a crosslinked rubber.

As indicated, the transducer 52 has a body 54 which encloses and holds a device used to convert energy into an ultrasonic vibration, above the range of human hearing. In one illustrative embodiment, the transducer body 54 includes a piezoelectric element, to convert electrical energy into sonic vibrations, and a final matching layer defining a transducer face. A piezoelectric crystal, which will oscillate at very high frequencies when an alternating current (AC) is applied across it, comprise the piezoelectric element. However, it is envisioned that any sort of ultrasonic transducer, including the types well known to those of ordinary skill in the ultrasound or the electrical arts, be employed as the transducer 52 encompassed by the transducer body 54. Additionally, as used herein, the term transducer body is broad and inclusive and includes embodiments where a transducer and its housing are initially separate components as well as embodiments where the transducer and housing therefor are one unitary structure from the time of assembly.

The front face 56 of the transducer 52 typically includes a matching layer that is lapped to provide a particular impedance. Advantageously, the lapping of the matching layer of the front face 56 can be completed to match the impedance of the elastomeric pad 70 included with the transducer system 50. In some transducers 52, the control system (not shown) provides fault detection in the event the front face 56 is not in contact with skin 24. If the ultrasonic transducer system 50 is to be used without modifying the transducer 52 and associated control system, the thickness of the elastomeric pad 70 needs to be kept to a minimum to prevent the elastomeric pad 70 from providing a false indication that the transducer 52 is in contact with skin 24 when it is not. For example, for one sample transducer 52, an elastomeric pad 70 formed from SEBS be less than about 2 millimeters thick and is preferably about 1.5 millimeters thick.

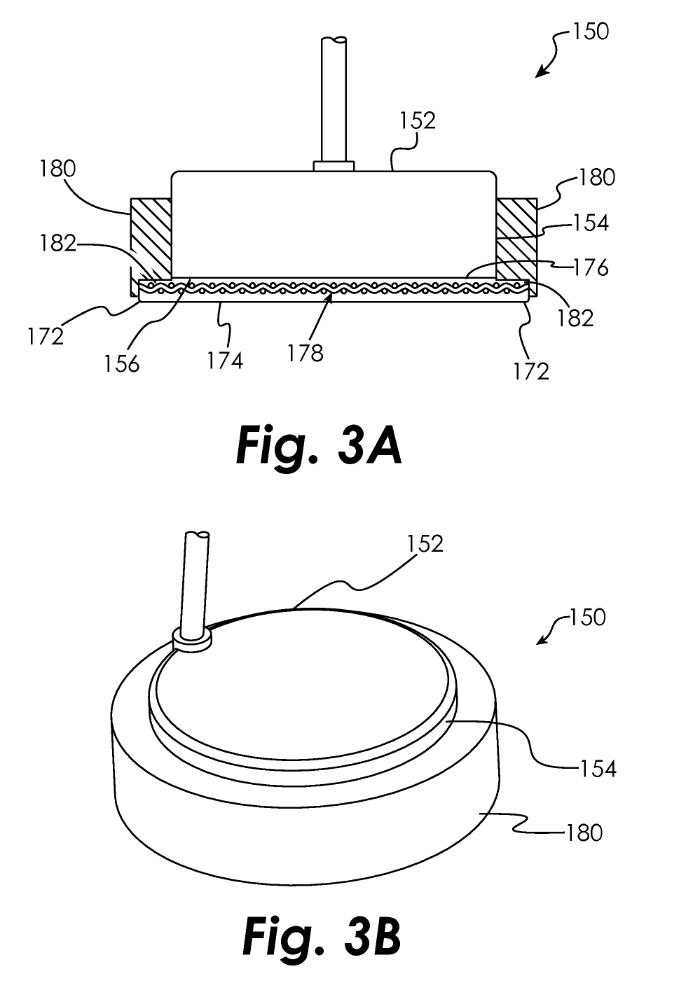

FIGS. 3A and 3B illustrate another illustrative ultrasonic transducer system 150 according to the present invention. The ultrasonic transducer system 150 also includes an ultrasonic transducer 152 and an elastomeric pad 170 [Note: there is no item #170 in diagram 3]. The elastomeric pad 170 includes a lip 172 that axially protrudes adjacent the perimeter of the front face 174. The elastomeric pad 170 includes a screen 178 that provides added structural integrity and bonds 182 to an adapter ring 180. Such an arrangement is thought to provide sufficient durability for multiple uses, potentially at least 300 treatments. The adapter ring 180 is coupled with the body 154 of the transducer 152.

The screen 178 can be formed from woven cloth, woven or molded polyester, or other material providing a support structure with minimal acoustic dissipation. In one illustrative embodiment the screen 178 is 57 mesh polyester, 400 microns thick, having a thread diameter of 143 microns, and 46% open area. A larger open area percentage be desirable; however, the open are is also a compromise between structural integrity and minimizing attenuation caused by the screen 178. In one illustrative embodiment, the screen 178 includes an open area percentage which increases from the perimeter to the center of the elastomeric pad 170. In one illustrative embodiment, the open area of the screen 178 is greater than 50%, and the screen 178 is formed from a finer wire than is typically available for the given open area percentage.

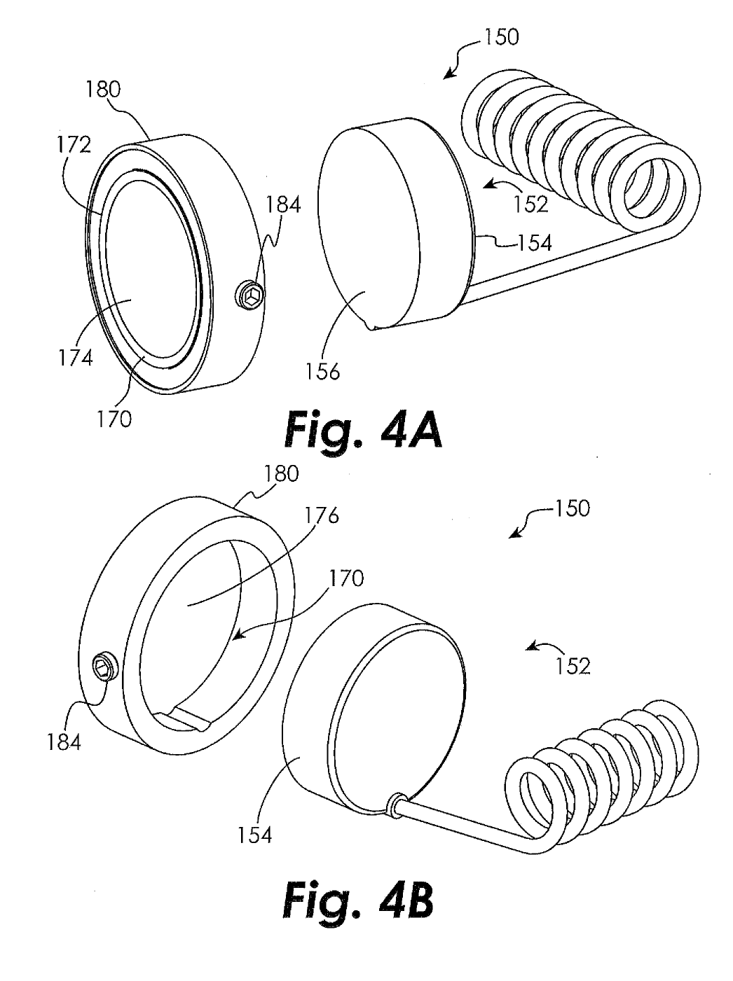

Referring to FIGS. 4A-4B, the elastomeric pad 170 and adapter ring 180 is shown separated from the ultrasonic transducer 152. With this configuration, advantageously an off-the-shelf ultrasonic transducer 152 can be equipped with an elastomeric pad 170 according to the present disclosure. If desired, the adapter ring 180 is fixed to the transducer body 152, for example in a set screw 184.

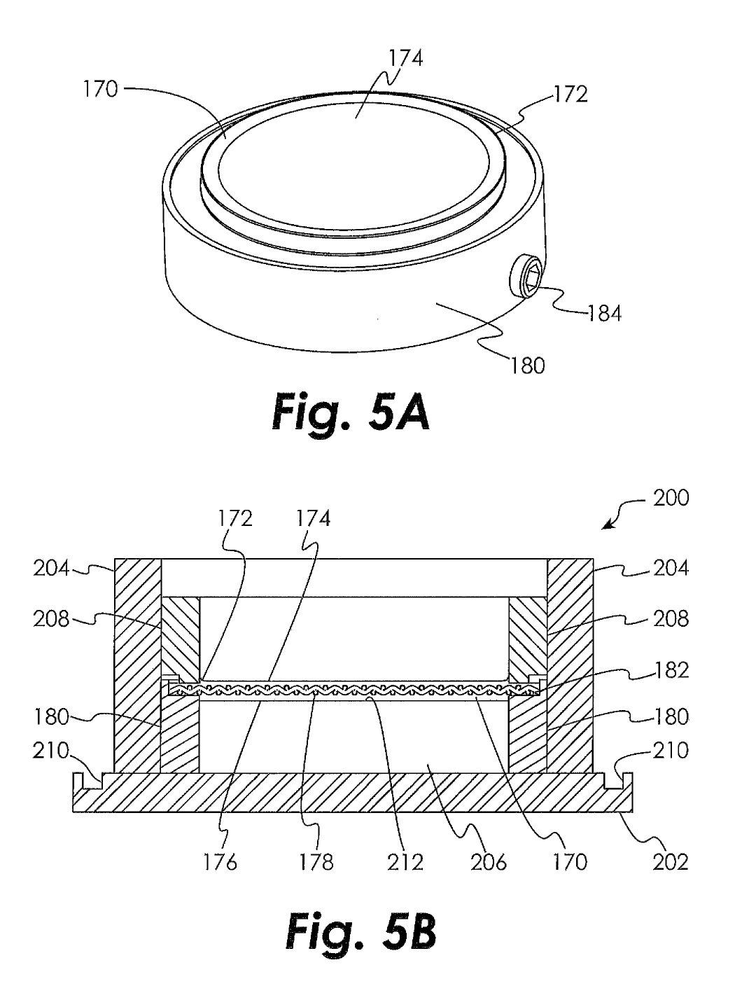

Referring to FIG. 5A, the elastomeric pad 170 and adapter ring 180 of the illustrative ultrasonic transducer system of FIG. 3A can be cast using a casting fixture 200 such as that shown in the cross-section view of FIG. 5B and the below-described processes.

Preparation of SEBS Material Slug. In developing the below process, some of the obstacles overcome include avoiding development of voids in the material because, once voids have formed, it is very difficult to remove them. Also the technique avoids handling steps which might cause the material to form strings:

Measure raw ingredients of three parts of DS-302 and one part of mineral oil (USP grade).

Mix an 80 gram batch of raw ingredients in a heavy aluminum casting bowl provided with a lid with a large vent hole; after mixing, pat flat and place in a vacuum for about 2 minutes to remove air from mixture.

Pre-heat hot-plate to a set temperature of about 200° C.

Position the casting bowl on the hot plate and monitor the temperature at the interior bottom of the hot-plate with a thermocouple.

Heat the mix to about 150° C., the recommended melt temperature of the DS-302 product.

During the heating, stir the mix repeatedly; the molten mix has a high viscosity at 150° C.

When the mix is uniformly melted, let the mix rest unstirred in the bowl for about 4 minutes to allow air bubbles to rise out of the melt.

Position the hot casting bowl in a vacuum jar with the lid in place and provide four brief pulls of vacuum to remove air from the mix, stopping when foam reaches the vent-hole.

Return the casting bowl to the hot-plate for approximately 3 additional minutes, then it transfer to a low-mass honeycomb setter and allow to slowly cool until solid, providing uniformity to the material and time for any remaining air bubbles to escape.

Preparation of the Matrix. The perimeter of screen 178 can be bonded to the ring 180 at bonding recess 182 formed in the inner portion of the top of the ring, for example:

Clean the adaptor ring 180 ultrasonically and in methanol, then prime with Lord AP134.

Apply a 3:1 blend of Loctite 0151 and Hysol epoxy to the perimeter of the screen 178.

Position the screen 178 on the ring 180, and hold in place with a weight (not shown).

Remove any excess epoxy with a flat edge tool and allow to cure overnight.

Mold for Casting the Elastomeric Pad. An illustrative mold 200 can be used for casting the elastomeric pad 170 on the screen 178 that is now bonded to the adapter ring 180. The mold 200 includes a base 202, an alignment fixture 204, an inner mold 206, and a stepped ring 208. The gutter 210 defined in the perimeter of the base 202 is used to prevent any couplant compound or mold release oil from leaking onto the hotplate (not shown). The alignment fixture 204 maintains concentricity of the adapter ring 180 and stepped ring 208 during molding of the elastomeric pad 170. The stepped ring 208 overlays the bonded perimeter area of the screen 178, thus limiting the formation of the front face 174 to where the screen 178 has open mesh. The inner mold 206 has an upper mold surface 212 that is concave, thus giving the back face 176 of the elastomeric pad 170 a convex contour, for example about 0.5 millimeters maximum height. The convex back face 176 is desirable because it reduces the likelihood of trapped air between the back face 176 and front face 156 (FIG. 3A) of the ultrasonic transducer 152. In one illustrative ultrasonic transducer system 150, the adapter ring 180 and the components of the mold 200 are formed from aluminum; however, other materials be utilized, although it has been noted that the material forming the elastomeric pad 170 bubbles in contact with the adaptor ring 180, for example when phenolic was used.

Casting the Elastomeric Pad. With the SEBS material cast into a void-free gel, and the mold 200 prepared, the elastomeric pad 170 can be molded onto the screen 178:

Set the hot plate temperature at about 175° C. and allow temperature to stabilize.

Cut an SEBS material slug, for example, with weight equal to 1.1 grams.

Clean the components of mold 200 with lint-free paper dipped in mineral oil.

Position the inner mold 206 on mold base 202, as shown in FIG. 5B.

Position the SEBS slug directly on inner mold 206 concave surface 212.

Position the adaptor ring 180 and screen 178 assembly, outer alignment ring 204, and stepped ring mold 208 as shown in FIG. 5B.

Place the assembled mold 200 components base on a hot plate.

Place a heat retaining cover (not shown) over alignment ring 204.

Place an outer aluminum foil heat retaining shield over mold 200.

Check the state of SEBS material slug at about 8 minutes. When flow is complete, remove mold 200 from hot plate and place on a honeycomb setter (not shown) to allow slow, even cooling.

Allow to cool to below 100° C. before cooling with water and removing the elastomeric pad 170 and adaptor ring 180 assembly.

Advantageously, the surface tension of the melted SEBS material forms a meniscus, referred to herein as a lip 172, around the perimeter of the face surface 174 of the elastomeric pad 170 at the junction with the stepped ring 208. As discussed above, this circumferential lip 172 provides a liquid seal to trap liquid between the face surface 74 and target surface such as skin 24. The trapped liquid can comprise both applied liquid to improve the acoustic coupling, such as water, and moisture released by the skin 24, which also improves acoustic coupling. Preferably, the thickness of elastomeric pad 170 is 1.5 mm or less.

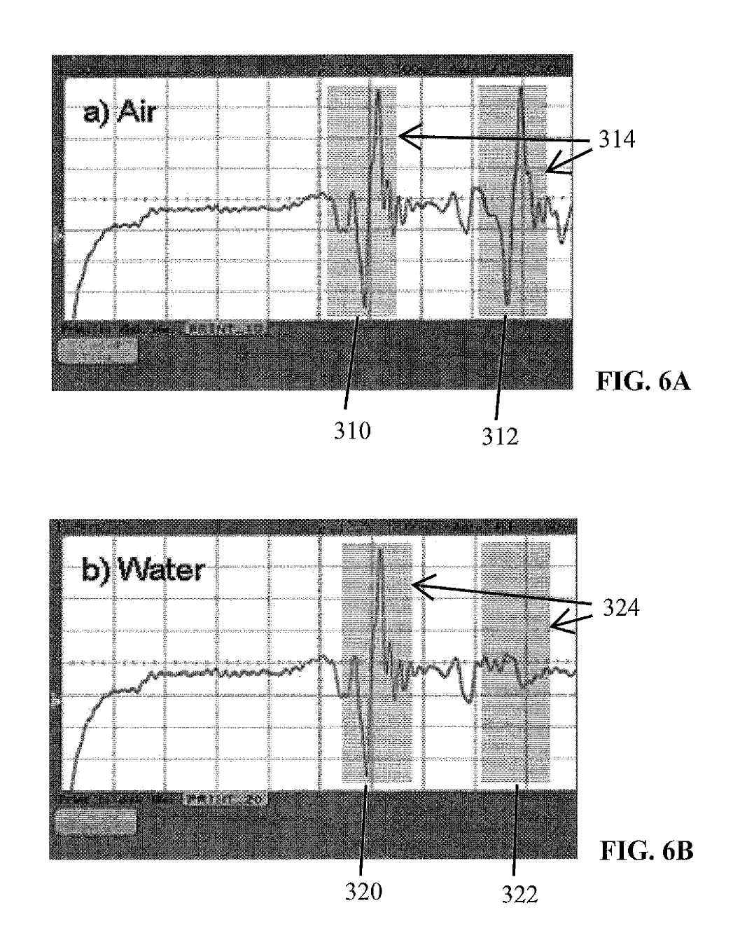

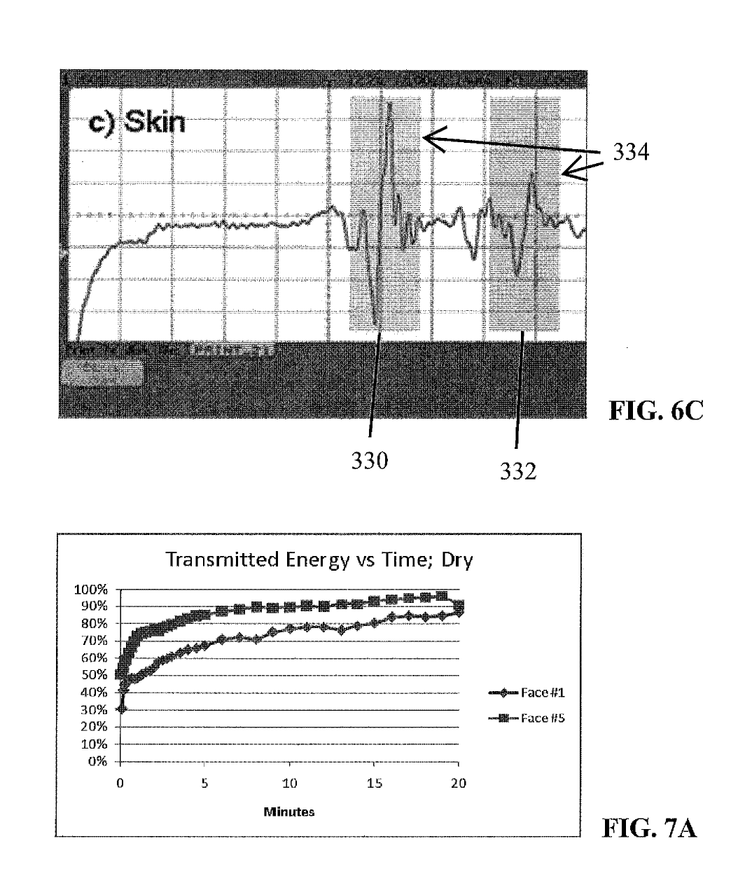

FIGS. 6A-6C illustrate acoustic wave reflection test results for the illustrative ultrasonic transducer system of FIG. 3A as coupled to air, water, and skin, respectively. To facilitate eliminating any piezoelectric ringing from the test measurements, a time delay line coupling was added between the transducer face 76, 176, and the elastomeric pad 70, 170. By comparing the amplitudes of the first and second reflections of the acoustic wave generated by the piezoelectric element (not shown) of the ultrasonic transducer 50, 150, the efficiency of the transmission of the acoustic wave for different final couplants to the target 26 can be evaluated. The first reflected acoustic wave reflected back to the piezoelectric element is from the delay line coupling interface with the elastomeric pad 70, 170. The second reflected acoustic wave reflected back is from the elastomeric pad 70, 170 interface with the air, water, or skin. Thus, the less the amplitude of the second reflected acoustic wave, the better the transmission of the acoustic wave out of the elastomeric pad 70, 170 and toward the target 26.

With the assumption that the acoustic wave that is not reflected back is transmitted, and given that acoustic energy is proportional to the square of the amplitude of acoustic wave, the acoustic energy transmitted through the air, water, or skin and on to the target 26 can be calculated from the difference between the first and second reflected acoustic wave amplitudes.

As can be noted from FIG. 6A, air is a poor acoustic wave couplant, and the amplitude 314 of the second reflection 312 is similar to that of the first reflection 310, because little to no acoustic wave energy is transmitted into the air and toward the target 26. As can be noted from FIG. 6B, water is an excellent acoustic wave couplant so the amplitude 324 of the second reflection is a small fraction 322 of that of the first reflection 320, indicating that most of the acoustic wave energy is transmitted through the water. As can be noted from FIG. 6C, because of hair and trapped air, dry coupling to skin 24 results in a amplitude 334 of the second reflection 332 that is about one half that of the first reflection 330, indicating that about three-quarters of the acoustic wave energy is transmitted into the skin 24 and toward the target 26. (Note that energy is proportional to amplitude squared, so if the reflected amplitude drops in half then reflected energy drops to one quarter.)

The illustrative ultrasonic transducer system 150 was tested when coupled with and without water. The test measurements were made either placing the transducer in contact with a dry and hairy patch of forearm, or with the patch wetted with a few drops of water. Transmitted energy was calculated by measuring the amplitude of the wave reflected back to the transducer and noting that the transmitted energy of a wave is proportional the square of the amplitude and with the assumption that the energy which was not reflected back was transmitted.

The test results illustrate that there is a significant advantage to having an elastomeric pad 170 and to trapping water or another coupling liquid between the elastomeric pad 170 and skin 24. Three illustrative ultrasonic transducer systems 150 were tested, one without an elastomeric pad 170, one with an elastomeric pad 170 having no perimeter lip 172, and one with an elastomeric pad 170 having a perimeter lip 172 that protrudes axially about 1 millimeter above the rest of the front face 174.

The test results show some consistent trends, in all cases the transmitted acoustic energy increased substantially when the skin was wetted:
a) no pad: 5.5% vs. 88.2%;
b) pad, but no lip: 75.3% vs. 93.4%; and
c) pad with lip: 71.0% vs. 99.5%.

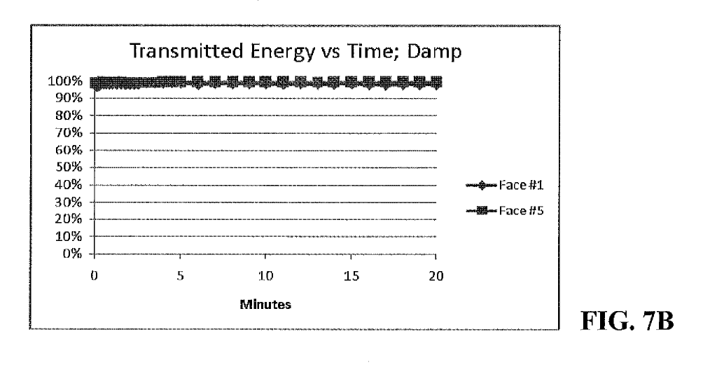

FIGS. 7A-7B illustrate test results for transmitted energy versus time for a illustrative ultrasonic transducer system 150 having an elastomeric pad 170 with a perimeter lip 172. FIG. 7A illustrates that although coupling and transmission improve over time when the front face 174 of elastomeric pad 170 begins dry, the best transmission is not achieved until 15 to 20 minutes have elapsed. In contrast, as shown in FIG. 7B, when the front face 174 of elastomeric pad 170 is wetted with water, the transmitted energy is immediately near 100% and remains steady over at least 20 minutes elapsed time. It is thought that the improvement realized over time in FIG. 7A is due not only to improved compliance of the front face 174 with the skin 24 over time, but also because skin 24 emits moisture, thus replacing the trapped air with perspiration.

Additionally, the transmitted acoustic energy under dry conditions for each structure with an elastomeric face was greater than ten times that for the transducer with no elastomeric face under dry conditions: 75.3% and 71% vs. 5.5%. The addition of the perimeter lip somewhat decreased the transmitted acoustic energy when the face was dry: 71.0% vs. 75.3%, but it also increased the transmitter acoustic energy when the surface was wetted: 99.5% vs. 93.4%.

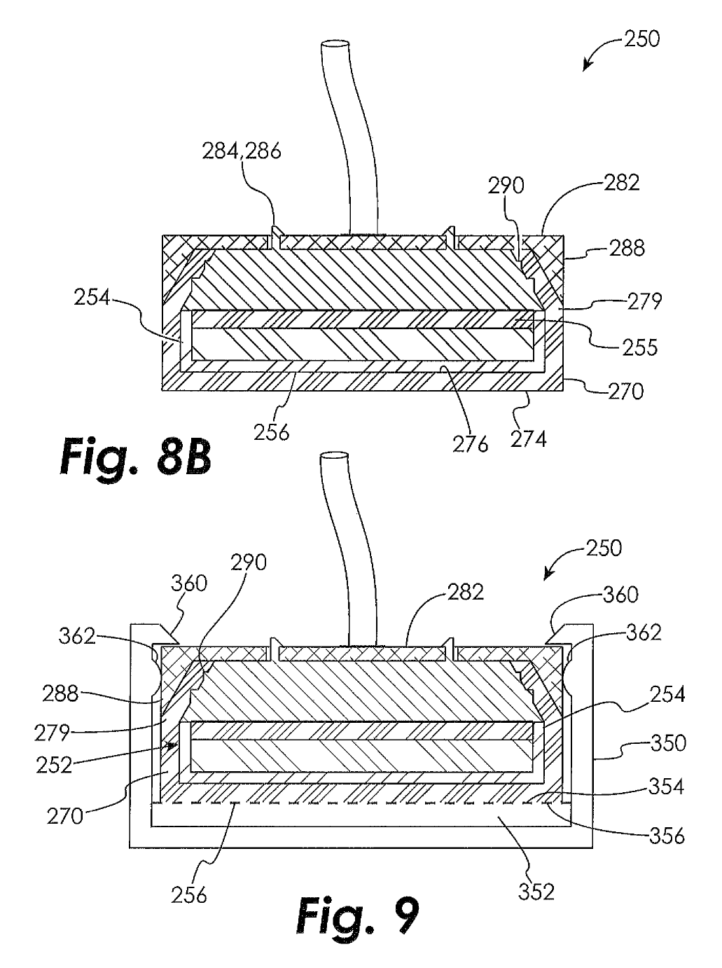

FIGS. 8A-8B respectively illustrate an exploded and an assembled cross-sectional view of another illustrative ultrasonic transducer system 250 according to the present disclosure. The elastomeric pad 270 advantageously includes an upwardly protruding flange(s) 279 for coupling the pad 270 to the body 254 of the ultrasonic transducer 252. The flange 279 be a continuous circular wall projecting upwardly from the perimeter of the back face 276, or be one or more individual flanges protruding upward from one or more arcs of the perimeter of the back face 276. The flanges be the same material, for example SEBS forming the lower portion between faces 274 and 276, or be defined by another material bonded or molded with the lower portion, such as the matrix screen 178 associated with the elastomeric pad 170 of the transducer system 150.

The elastomeric pad 270 include a downwardly projecting lip 272. Additionally or alternatively, the elastomeric pad 270 include the front face 274 defining a concave contour. Also, the upper surface 276 define a convex contour.

The ultrasonic transducer 252 includes a front face 256, a transducer body 254, a piezoelectric element 255, a receiver 280, and a locking cap 282. The receiver 280 and locking cap 282 include features to provide coupling of flange 279 to the transducer body 254. For example, illustrative receiver 280 includes protuberances 290 and locking posts 286. As shown in FIG. 8B, when the back face 276 of the pad 270 is in contact with the front face 256 of the ultrasonic transducer 252, flange 279 is positioned over protuberances 290. Locking cap 282 is retained to receiver 280 of the transducer 252, for example, via retaining posts 286 interlocking with openings 284 defined by cap 282. Advantageously, when locking cap 282 is locked to receiver 280, downward projections 288 compresses flange 279 against receiver 280 and protuberances 290, thus binding elastomeric pad 270 to the ultrasonic transducer 152. The flange 279 be in tension, thereby holding the back face 276 of the pad 170 tight against the front face 276 of the ultrasonic transducer 252.

FIG. 9 illustrates a cross-sectional view of the illustrative ultrasonic transducer system 250 of FIGS. 8A-8B contained within a rewetting cup 350 according to the present disclosure. Rewetting cup 350 contains a rewetting solution that renews the moisture content of the elastomeric pad 270, such as mineral oil, thereby extending the useful life of the elastomeric pad 270. The rewetting cup 350 also include an absorbent pad 352 for retaining the rewetting solution. The cup 350 and rewetting pad 352 can be arranged so that the transducer system 250 be positioned with the front face 256 of the elastomeric pad 270 in contact with the rewetting pad 270. The cup 350 also include a filter 354 positioned between the elastomeric pad 270 and the rewetting pad 352, for example, a filter 354 that provides unidirectional flow of rewetting solution from the rewetting pad 352 to the elastomeric pad 270. The cup 350 also include one or more retaining members 360 for retaining the transducer system 250 and compressing the elastomeric pad 250 against the rewetting pad 352. The cup 350 also include a seal 362 for preventing the solution from evaporating or otherwise escaping the cup 350.

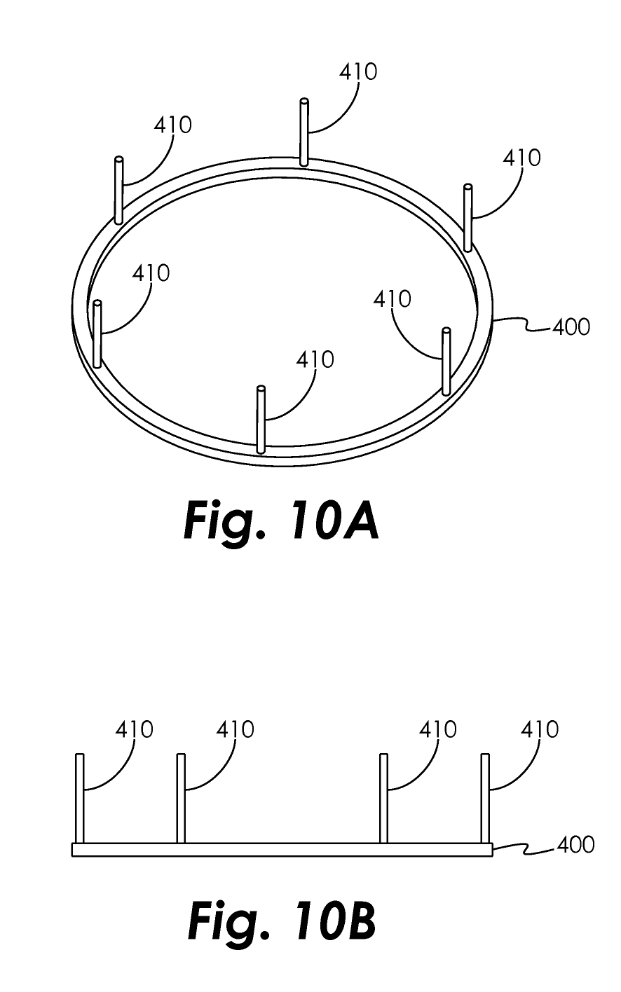

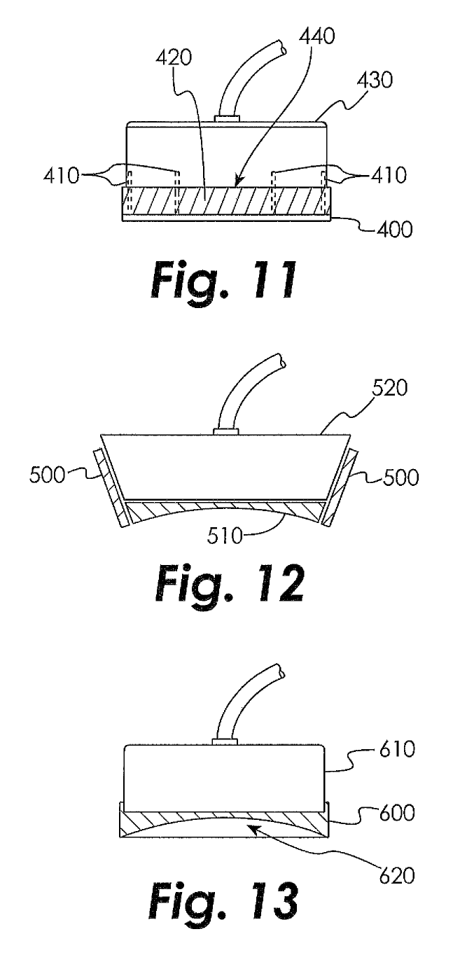

There are additional illustrative devices for connecting an elastomeric pad to an ultrasonic transducer using adapters, and one such illustrative device is depicted in FIGS. 10A-10B. Used in lieu of a screen, the adapter ring 400 of FIG. 10A includes a plurality of pins 410 configured to be generally orthogonal to the ring 400. The pins 410 initially be separate components attached to the ring 400 or be integral with the body of the ring 400 and manufactured from an injection mold as a single piece. Illustratively, the ring 400 and the pins 410 are manufactured from a thermoplastic, such as the commonly-encountered acrylonitrile butadiene styrene (ABS), although other solids are envisioned, including ceramic materials. In one embodiment, six pins 410 are used, and they are composed of stainless steel posts 0.0036″ in diameter. If not initially molded as part of the ring 400, the pins 410 are attached to the ring 400 by compression setting: undersized holes, slightly smaller than the diameter of the pins 410, are formed in the ring 400 using a drill press, and the pins 410 are positioned in the ring 400 holes and fixed in place through the application of pressure. FIG. 11 shows an assembly consisting of the ring 400, an elastomeric pad 420, and a transducer body 430. The pins 410 on the ring 400 engage and mate with corresponding apertures cut through the elastomeric pad 420 and apertures cut in the face 440 of the transducer body 430.

FIG. 12 shows an alternate illustrative, adapter-type of device, namely a sleeve 500, for connecting an elastomeric pad 510 to an ultrasonic transducer body 520. The shape of the sleeve 500 is a generally downward-pointing conic section, permitting a simple assembly of dropping the elastomeric pad 510 and the ultrasonic transducer body 520 therein and the use of use friction or pressure to retain the components in place. Similar to the adapter ring, the sleeve 500 be composed of any number of solids known in the art, including ABS and ceramic materials.

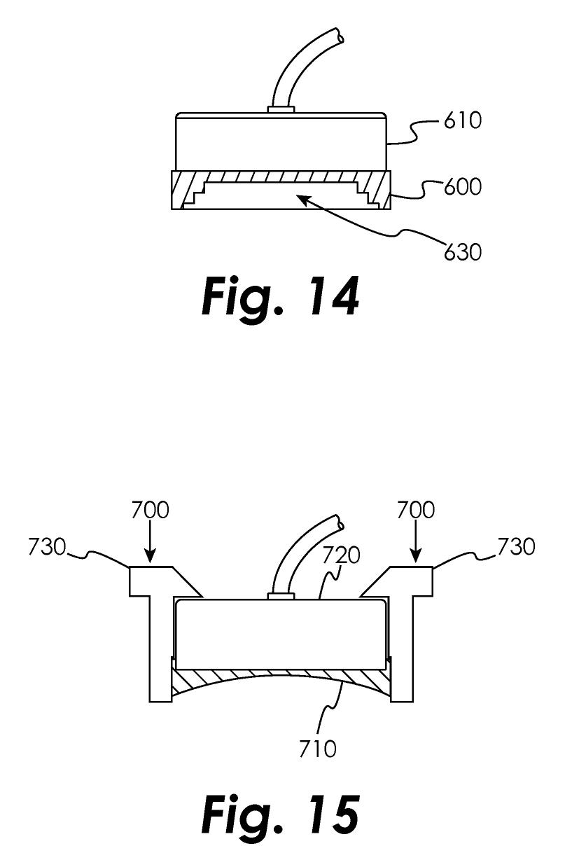

As noted above, the illustrative elastomeric pad has a recess on its front face designed to trap a liquid. FIG. 13 shows an elastomeric pad 600, coupled to a transducer body 610, which has a concave recess 620 capable of trapping a liquid couplant, for example, water, between the pad 600 and a target surface such as skin. One alternative contour for this elastomeric pad structural feature is the stepped recess 630 shown in FIG. 14. The stepped recess 630 is similarly capable of trapping a liquid couplant between an elastomeric pad 600 and a target surface.

It is envisioned that a replaceable elastomeric pad be used with the ultrasonic transducer system disclosed and claimed herein. To that end, the illustrative embodiment depicted in FIG. 15 includes at least one adapter clip 700 which is used to releaseably attach an elastomeric pad 710 to a transducer body 720. Tabs 730 at the ends of the clips 700 distal from the transducer body 720 assist with the connection and removal process by their ability to at least slightly move away from the transducer body 720 in response to downward pressure. In this embodiment, the clips 700 are periodically spaced and do not form a complete lip or seal around the perimeter of the transducer body 720. The clips 700 be composed of the same material as the elastomeric pad 710, such as SEBS, or be composed of any semi-elastic material capable of returning to its original shape after stress applied to it is removed.

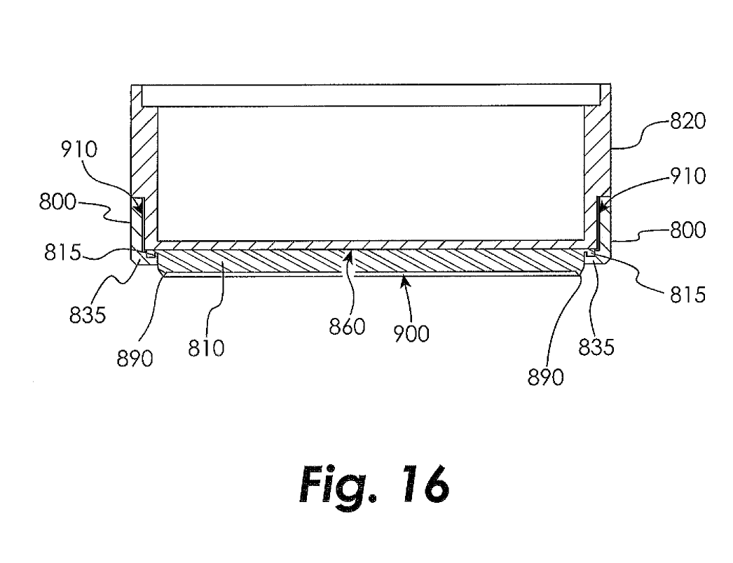

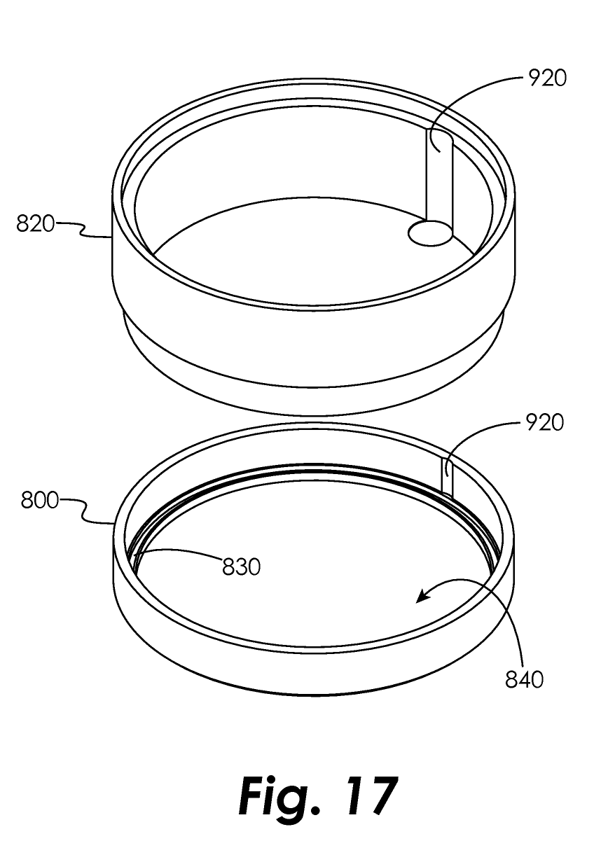

In yet another embodiment of the present invention, the elastomeric pad be bonded to the transducer body. Acceptable adhesives to accomplish this bonding include, but are not limited to, a heat-cured epoxy bond and other commercial bonding materials known to those in the art. Further, an adapter ring 800, as illustrated in FIGS. 16 and 17, be used in lieu of pins or other means for connecting the elastomeric pad 810 to the transducer body 820. The adapter ring 800 is adhered or otherwise affixed to at least a segment of the exterior of the transducer body 820, which as noted be a separate housing component, using a bond such as an epoxy adhesive or other means for connecting synthetic materials known in the art. An exemplary adapter ring 800 is contoured to engage and retain an exterior surface 815 of the elastomeric pad 810. For example, in one embodiment, one or more grooves 830 or channels on the adapter ring interior 840 correspond to the contour of the exterior surface 815 of the elastomeric pad 810 and can thereby couple the elastomeric pad 810 and the transducer body 820. A lip 835 comprise part of the adapter ring 810 and be contoured to engage a corresponding lip or at least some perimeter segment of the exterior surface 815 of the elastomeric pad 810, in order to couple the elastomeric pad 810 and the transducer body 820. The adapter ring lip 835, in one illustrative embodiment, comprise part of the recess which holds a liquid couplant between the elastomeric pad 810 and a target surface.

The adapter ring 800 is preferably manufactured from an inert synthetic compound such as silicone, polypropylene or nylon, but be composed of any of a wide variety of polymers known in the art which are capable of forming a liquid seal. It should be noted that, while a circular adapter ring 800 is used in one embodiment, it is envisioned that a wide variety of adapter shapes be used, so long as at least a section of the shape of the adapter ring 800 and a section of the shape of the transducer body 820 are generally equal in diameter and capable of mating.

In one embodiment, the elastomeric pad 810 is molded to have a convex curvature on its back face 860. When pressed against the flat transducer body 820 during system assembly, the convex shape is eliminated and hence is not visible in FIG. 16. During assembly, this initially-convex shape tends to force air present between the elastomeric pad 810 and the transducer body 820 toward the outer periphery of those two stacked components and out through ventilation groove 920 (FIG. 17). Air is a very poor acoustic wave couplant, and the elimination of any air caught between the elastomeric pad 810 and transducer body 820 is therefore quite desirable.

The front face of the elastomeric pad 810 have an annular lip 890 which defines a recess 900 capable of retaining a liquid couplant such as water between the elastomeric pad 810 and the target surface. As be seen in FIG. 16, the retaining lip 890 be cross-sectionally triangular in shape, with the bottom of the lip coming to a point which abuts the target surface. Both the transducer body 820 and the adapter ring 800 have a slight inward taper 910, 2° in one embodiment, to assist with the removal of these parts from a mold.

Referring to FIG. 17, one aspect of an illustrative embodiment of the present invention is a device for removing any air trapped, at the time of assembly or otherwise, between the elastomeric pad 810 and the transducer body 820, thereby improving performance of the system through removal of a poor couplant. As indicated, the initially-curved top of the elastomeric pad 810 serves to flush, generally laterally in direction, any air which happens to be caught between the transducer body 820 and elastomeric pad 810 to the interior 840 of the adapter ring 800 as the elastomeric pad 810 is pressed against the transducer body 820 during assembly. A small ventilation slot or groove 920, vertical in one illustrative embodiment, is present and aligned in both the elastomeric pad 810 and the transducer body 820, and the groove 920 then channels the air out of the system.

The various illustrative embodiments of the gel-free ultrasonic transducer system discussed herein be practiced as a method for improved ultrasonic transducer transmission. The initial step for this method entails coupling an ultrasonic transducer body, which includes a piezoelectric element and a layer comprising a transducer face, with an elastomeric pad, which comprises a back face in direct contact with the transducer face and a front face defining a recess. Once the transducer body and elastomeric pad are coupled, they are to be positioned over an extradermal target surface, such as over a window cut in a bone fracture cast worn by a medical patient, with the elastomeric pad generally abutting and in contact with the target surface. The use of ultrasound in medical applications, such as the promotion of bone growth or to treat arthritis, is well documented, but the claimed system and method be practiced in virtually any situation requiring superior propagation of an ultrasound signal. As noted, the elastomeric pad in one illustrative embodiment of the system includes a transdermal pharmaceutical which is delivered by contact with skin and the stimulation of flow that results from its interaction with ultrasound waves.

While the present invention has been illustrated and described in detail in the foregoing drawings and description, the same is to be considered as illustrative and not restrictive in character, it being understood that only illustrative embodiments thereof have been shown and described and that all changes and modifications that come within the spirit and scope of the invention as defined in the following claims are desired to be protected.

1. An ultrasonic transducer system comprising:
a transducer body comprising
a piezoelectric element, and
a layer defining a transducer face; and
an elastomeric pad comprising
a back face in direct contact with the transducer face; and
a front face defining a recess.

2. The ultrasonic transducer system of claim 1, wherein the elastomeric pad is formed from the elastomeric copolymer styrene-ethylene/butylene-styrene (SEBS), silicone, or silicone rubber.

3. The ultrasonic transducer system of claim 1, wherein the elastomeric pad front face recess is concave.

4. The ultrasonic transducer system of claim 1, wherein the elastomeric pad front face recess is stepped.

5. The ultrasonic transducer system of claim 1, wherein the elastomeric pad is porous.

6. The ultrasonic transducer system of claim 1, wherein the transducer face is lapped to provide a particular impedance.

7. The ultrasonic transducer system of claim 1, wherein the elastomeric pad has an acoustic impedance generally matching that of human skin.

8. The ultrasonic transducer system of claim 1, wherein the elastomeric pad is shaped to provide coupling with the transducer body.

9. The ultrasonic transducer system of claim 1, further comprising one or more coupling members releasably coupling the elastomeric pad to the transducer body.

10. The ultrasonic transducer system of claim 1, further comprising a cap shaped for releasable attachment to the transducer body to thereby cover the elastomeric pad.

11. The ultrasonic transducer system of claim 7, wherein the cap comprises an interior having a rewetting structure shaped to engage the front face of the elastomeric pad when the cap is attached to the transducer body.

12. The ultrasonic transducer system of claim 8, wherein the rewetting structure contains a rewetting solution.

13. The ultrasonic transducer system of claim 1, wherein the rewetting structure further comprises a filter.

14. The ultrasonic transducer system of claim 1, wherein the elastomeric pad comprises a screen bonded to a support structure coupled to the transducer body.

15. The ultrasonic transducer system of claim 15, wherein the support structure has an interior recess into which the perimeter of the elastomeric pad is bonded.

16. The ultrasonic transducer system of claim 1, wherein the elastomeric pad comprises a transdermal pharmaceutical.

17. The ultrasonic transducer system of claim 1, wherein an adapter is used to couple the elastomeric pad to the transducer body.

18. The ultrasonic transducer system of claim 17, wherein the adapter is a ring having one or more pins which couple the elastomeric pad and the transducer body.

19. The ultrasonic transducer system of claim 17, wherein the adapter is a ring affixed to the transducer body and contoured to engage an exterior surface of the elastomeric pad, thereby coupling the elastomeric pad and the transducer body.

20. The ultrasonic transducer system of claim 17, wherein the adapter is a sleeve which retains the elastomeric pad and the transducer body.

21. The ultrasonic transducer system of claim 17, wherein the adapter and the transducer body include a channel through which air trapped between the elastomeric pad and transducer body is released.

22. The ultrasonic transducer system of claim 1, wherein the elastomeric pad is adhesively bonded to the transducer body.

23. The ultrasonic transducer system of claim 1, wherein the elastomeric pad is replaceable.

24. A method for ultrasonic transducer transmission comprising the steps of:
providing a transducer comprising:
a transducer body having a transducer face,
a piezoelectric element, and
an elastomeric pad comprising
a back face and
a front face defining a recess, such that the elastomeric pad back face is in direct contact with the transducer face; and
providing a liquid couplant to the recess of the elastomeric pad;
positioning the transducer over a target surface so that the elastomeric pad and liquid couplant abut the target surface; and
generating an ultrasonic wave from the piezoelectric element such that the wave is transmitted through the elastomeric pad to the target surface.

25. A method for promoting bone growth in a living being, using an ultrasonic transducer system of claim 1, comprising:
providing an ultrasonic transducer system of claim 1,
applying a liquid couplant to the recess of the elastomeric pad,
touching the elastomeric pad and to the body of a living being, and
transmitting an ultrasonic wave from the transducer body, through the piezoelectric elastomeric pad and couplant, to the living being.

26. The method of claim 25, wherein the living being has a cast having an aperture, and the transducer elastomeric pad is inserted into the aperture to touch the living being.
